(19) United States
(12) Patent Application Publication
(10) Pub. No.: US 2024/0100107 A1
(43) Pub. Date: Mar. 28, 2024

(54) SENECA VALLEY VIRUS COMBINATION THERAPY TO TREAT A CANCER REFRACTORY TO A CHECKPOINT INHIBITOR

(71) Applicant: Seneca Therapeutics, Inc., Blue Bell, PA (US)

(72) Inventors: Paul L. HALLENBECK, Doylestown, PA (US); Sunil CHADA, Houston, TX (US)

(21) Appl. No.: 18/271,619

(22) PCT Filed: Jan. 11, 2022
(86) PCT No.: PCT/US2022/011994

§ 371 Date: Jul. 10, 2023

Related U.S. Application Data
(60) Provisional application No. 63/135,914, filed on Jan. 11, 2021.

(51) Int. Cl. A61K 35/768; A61K 9/00; A61P 35/00; C07K 16/28

(52) U.S. Cl. CPC A61K 35/768; A61K 9/0019; A61P 35/00; C07K 16/2818; A61K 2039/507

(57) ABSTRACT

Provided herein are compositions and methods of using Seneca Valley Vims (SVV) or SVV derivative in combination with a checkpoint inhibitor for treating a cancer that is refractive to treatment by the checkpoint inhibitor. Also provided herein are kits containing a Seneca Valley Vims (SVV) or SVV derivative and a checkpoint inhibitor for treating a cancer that is refractive to treatment by the checkpoint inhibitor.

### CROSS-REFERENCE TO RELATED APPLICATIONS

This application claims priority to U.S. Provisional Application No. 63/135,914, filed Jan. 11, 2021, the disclosure of which is incorporated by reference in its entirety.

### TECHNICAL FIELD

The disclosed inventions relate to compositions and methods for treating cancer. More particularly, the disclosed inventions relate to the field of treating cancer in a subject using an oncolytic virus, in particular Seneca Valley Virus (SVV) or SVV derivative, in combination with a checkpoint inhibitor.

### BACKGROUND

Cancer is the second most common cause of death in the United States. One out of every four individuals dies from it, and more than one million new cancer diagnoses are made every year. The disease begins with the uncontrolled proliferation and growth of abnormal, transformed cells. However, the definition does not end with a description of one disease but of hundreds of different diseases. No two cancers are the same, nor are they clonal. The mutations driving and bought during cell transformation may be similar, but they are never identical. This conundrum adds to the complexity and heterogeneity of the pathologies that patients develop. Current cancer therapies, including chemotherapeutics and radiation, are most effective when combined with immunomodulatory agents to create and enhance the antitumor microenvironment. Many malignancies may be resistant to treatment via these traditional methods.

Oncolytic viruses show enormous potential as anti-cancer agents. The picornavirus Seneca Valley virus (SVV) is a single stranded (+) RNA virus that has been investigated as an oncolytic therapy. It has been shown that SVV can target and facilitate regression of many intractable malignancies, including small and non-small cell lung cancers and pediatric solid tumors.

A frequent problem associated with cancer treatment is that a cancer may be refractory to a specific cancer therapy. The word “refractory” generally means stubborn or intractable, and in medicine it is specifically applied to disease that does not respond to treatment. Refractory cancer refers to cancer that may be resistant at the beginning of treatment or becomes resistant during treatment. For example, checkpoint inhibitors (a class of anticancer agents) are known to be refractory to neuroendocrine cancers and small cell lung cancer (SCLC) tumors.

Accordingly, what is needed is an improved therapeutic approach for using oncolytic viruses, in particular SVV, to treat cancers which have become or are refractory to monotherapy cancer treatment, by, for example a checkpoint inhibitor.

### SUMMARY

Provided herein are improved methods, compositions, kits, and pharmaceutical composition for treating cancer which use Seneca Valley Virus (SVV) or SVV derivative in combination with a checkpoint inhibitor, whereby the cancer is refractory to monotherapy with the checkpoint inhibitor. In particular, the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer. The cancer may also be a neuroblastoma, a melanoma, a neuroendocrine cancer, or a small cell lung cancer (SCLC) tumor. In certain embodiments, the SVV derivative encodes a checkpoint inhibitor. In other embodiments, the SVV derivative encodes the checkpoint inhibitor.

One embodiment of the invention is a method of treating a cancer in a subject in need thereof comprising administering to the subject an effective amount of Seneca Valley Virus (SVV) or SVV derivative, wherein the subject is also administered an effective amount of at least one checkpoint inhibitor, and whereby the cancer is refractory to monotherapy with the checkpoint inhibitor.

Another embodiment of the invention is a method of improving the success of oncolytic cancer virus treatment comprising administering an effective amount of Seneca Valley Virus (SVV) or SVV derivative to a subject having cancer, wherein the subject is also administered an effective amount of at least one checkpoint inhibitor, wherein the cancer treatment is improved when compared to a subject only having received the effective amount of the Seneca Valley Virus or SVV derivative, and whereby the cancer is refractory to monotherapy with the checkpoint inhibitor.

In the methods, the checkpoint inhibitor is administered before, concurrent or after administration of the Seneca Valley Virus (SVV) or SVV derivative.

Also provided herein is a pharmaceutical composition for treating a cancer in a subject, the pharmaceutical composition comprising a checkpoint inhibitor, an SVV or SVV derivative, and a pharmaceutically acceptable carrier, wherein the cancer is refractory to monotherapy with the checkpoint inhibitor.

Further provided herein is a kit for treating cancer in a subject comprising a Seneca Valley Virus (SVV) or SVV derivative combined with at least one checkpoint inhibitor, wherein the cancer is refractory to monotherapy with the checkpoint inhibitor.

Additionally, provided herein is a Seneca Valley Virus (SVV) or SVV derivative in combination with at least one checkpoint inhibitor for use in the manufacture of a medicament for treatment of a cancer, wherein the cancer is refractory to monotherapy with the checkpoint inhibitor.

In certain embodiments, the checkpoint inhibitor is a PD-1 inhibitor, a PD-L1 inhibitor, or a CTLA-4 inhibitor. In other embodiments, the checkpoint inhibitor is an anti-PD1-antibody, an anti-PD-L1 antibody, or an anti-CTLA-4 antibody. In certain embodiments, the checkpoint inhibitor blocks one or more of the following checkpoint proteins on cancer cells: PD-1, PD-L1, CTLA-4, B7-1, B7-2, The checkpoint inhibitor may be an antibody (e.g. a monoclonal antibody) or a nanobody. In certain embodiments, the SVV derivative encodes a checkpoint inhibitor. In other embodiments, the SVV derivative encodes the checkpoint inhibitor.

Other features and advantages of the invention will be apparent from the detailed description and examples that follow.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 shows the Pan02 Tumor Model Treatment schedule.

FIG. 2A shows the effects of administering SVV, checkpoint inhibitors and SVV and checkpoint inhibits in primary tumors based on tumor volume (mm³) as function of days post-inoculation.

FIG. 2B shows the effects of administering SVV, checkpoint inhibitors and SVV and checkpoint inhibits in contralateral tumors based on tumor volume (mm³) as function of days post-inoculation.

FIG. 3A, FIG. 3B, and FIG. 3C show a Kaplan Meier plot depicting the percentage survival of cells as a function of time after treatment with SVV, checkpoint inhibitors or combinations thereof.

FIG. 4 shows the tumor volume after treatment SVV, checkpoint inhibitors or combinations thereof based on tumor volume (mm³) as function of days post-inoculation (study days). SVV-001 treatment stopped at day 12. Anti-PD1/anti-CTLA4 treatment stopped at day 23.

FIG. 5A and FIG. 5B show the tumor volume after treatment SVV, checkpoint inhibitors or combinations thereof based on tumor volume (mm³) as function of days post-inoculation (study days).

### DETAILED DESCRIPTION OF THE INVENTION

The general description and the following detailed description are exemplary and explanatory only and are not restrictive of the invention, as defined in the appended claims. Other aspects of the present invention will be apparent to those skilled in the art in view of the detailed description of the invention as provided herein

The present invention relates to compositions and methods of using Seneca Valley Virus (SVV) or a derivative thereof for treating cancer in a subject. The SVV or SVV derivative is useful in a variety of applications such as treating a cancer, reducing, or inhibiting cancer cells growth, and increasing the survival of subject suffering from cancer. In particular, the present invention relates to composition and methods that use SVV or SVV derivative in combination with a checkpoint inhibitor in a patient that has cancer that is refractory to monotherapy with the checkpoint inhibitor.

This invention is based on the surprising discovery that use of SVV or SVV derivative in combination with a checkpoint inhibitor to treat a cancer, which is refractory to treatment by the checkpoint inhibitor, improves the success of cancer treatment. This invention is also based on the surprising discovery that when treating cancers SVV and checkpoint inhibitors alone do not elicit high cure rates in an animal (mouse) model of cancer nor a good abscopal effect (systemic anti-tumor immune response) but when two used together in conjunction both high cure rates and systemic anti-tumor effects are seen.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or +10%, more preferably +5%, even more preferably +1%, and still more preferably +0.1% from the specified value, as such variations are appropriate to perform the disclosed methods.

The term “biological” or “biological sample” refers to a sample obtained from an organism or from components (e.g., cells) of an organism. The biological sample may be obtained from tumor cells or tumor tissue. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein the term “Seneca Valley Virus” or “SVV” encompass wild type SVV or an SVV derivative. Exemplary suitable SSV strains include SVV-001, NTX-010, and the SVV strain having ATCC Patent Deposit Number PTA-5343. As used herein, the term “derivative” specifies that a derivative of a virus can have a nucleic acid or amino acid sequence difference in respect to a template viral nucleic acid or amino acid sequence. For instance, an SVV derivative can refer to an SVV that has a nucleic acid or amino acid sequence different with respect to the wild-type SVV nucleic acid or amino acid sequence of ATCC Patent Deposit Number PTA-5343. In some embodiments, the SVV derivative encompasses an SVV mutant, an SVV variant or a modified SVV (e.g. genetically engineered SVV). In one embodiment, the SVV derivative is a SVV virus modified to express a therapeutic protein. Examples of such SVV derivative may be found in U.S. Provisional Patent Application No. 63/138,999 filed on Jan. 19, 2021, the disclosure of which is incorporate herein to the extent it pertains to armed SVV constructs. Exemplary suitable SVV derivatives are the following SVV armed constructs: (1) SVV-001/Enhanced IL-2 (SVV-024); (2) SVV-001/Anti-PD-L1 (SVV-012); (3) SVV-001/CXCL9 (SVV-037); (4) SVV-001/TGF beta decoy (SVV-044); (5) SVV-001/Nitroreductase (SVV-058); (6) SVV-001/IL2-IL15 Fusion Protein (SVV-069); and (6) SVV-001/Ovalbumin epitope (SVV-077). These armed SVV constructs were designed by Seneca Therapeutics, Inc. In other embodiments, the SVV derivative may be the ONCR-788. In some embodiments, the modified SVV derivative is modified to be capable of recognizing different cell receptors or to be capable of evading the immune system while still being able to invade, replicate and kill the cell of interest (i.e. cancer cell). In other embodiments, SVV is modified to express an agent that is useful for treating cancer. In general, an SVV or SAN derivative can be derived from an already pre-existing stock of virus that is passaged to produce more viruses. SVV or SVV derivative can also be derived from a plasmid. The SVV derivative may encode a checkpoint inhibitor, such as a nanobody.

As used herein, “higher” refers to expression levels which are at least 10% or more, for example, 20%, 30%, 40%, or 50%, 60%, 70%, 80%, 90% higher or more, and/or 1.1-fold, 1.2-fold, 1.4-fold, 1.6-fold, 1.8-fold, 2.0-fold higher or more, and any and all whole or partial increments therebetween, than a control reference. A disclosed herein an expression level higher than a reference value refers to an expression level (mRNA or protein) that is higher than a normal or control level from an expression (mRNA or protein) measured in a healthy subject or defined or used in the art.

As used herein, “lower” refers to expression levels which are at least 10% lower or more, for example, 20%, 30%, 40%, or 50%, 60%, 70%, 80%, 90% lower or more, and/or 1.1-fold, 1.2-fold, 1.4-fold, 1.6-fold, 1.8-fold, 2.0-fold lower or more, and any and all whole or partial increments in between, than a control reference. A disclosed herein an expression level lower than a reference value refers to an expression level (mRNA or protein) that is tower than a normal or control level from an expression (mRNA or protein) measured in a healthy subject or defined or used in the art.

As used herein, the terms “control,” or “reference” can be used interchangeably and refer to a value that is used as a standard of comparison.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime. For purposes herein, a combination therapy can include a treatment regime that includes administration of an oncolytic virus and another anti-cancer agent, each for treating the same hyperproliferative disease or conditions, such as the same tumor or cancer. In preferred embodiments, the combination therapy includes administration of SVV or SVV derivative in conjunction with one or more checkpoint inhibitor in a patient.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to intra-tumoral, intravenous, intrapleural, oral, aerosol, parenteral, ophthalmic, pulmonary, and topical administration.

The language “pharmaceutically acceptable carrier” includes a pharmaceutically acceptable salt, pharmaceutically acceptable material, composition, or carrier, such as a liquid or solid filler, diluent, excipient, solvent, or encapsulating material, involved in carrying or transporting a compound(s) (e.g. SVV or SVV derivative and/or a checkpoint inhibitor) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “effective amount” or “therapeutically effective amount” means the amount of the virus particle or infectious units generated from vector of the invention which is required to prevent the particular disease condition, or which reduces the severity of and/or ameliorates the disease condition or at least one symptom thereof or condition associated therewith.

As used herein the phrase “cancer refractory to monotherapy” with the checkpoint inhibitor refers to any cancer that may be resistant at the beginning of treatment to monotherapy with a checkpoint inhibitor or SVV, or becomes resistant to monotherapy with a checkpoint inhibitor or SVV during treatment. The phrase includes cancers that have been treated with the checkpoint inhibitors but have not responded (i.e. are resistant to the cancer treatment). The phrase also includes cancers that have been treated with a checkpoint inhibitor and initially responded to the treatment, but subsequently the tumor regrows (relapsed/resistant). For the proposes of this definition, the term monotherapy with a checkpoint inhibitor refers cancer that have been treated with a checkpoint inhibitor as the only anti-cancer agent. Examples of such cancers include cold tumors, which are cancers that have not been recognized or have not provoked a strong response by the immune system. Cold tumors are resistant to checkpoint inhibitors and/or checkpoint blockage.

A “subject” or “patient,” as used therein, may be a human or non-human mammal. Non-human mammals include, for example, livestock and pets, such as ovine, bovine, porcine, canine, feline, and murine mammals. Preferably, the subject is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### SVV or SVV Derivative and Checkpoint Inhibitors to Treat Cancer Refractory to Checkpoint Inhibitor Monotherapy

The disclosure provides for methods, compositions, kits, and pharmaceutical composition for treating cancer which utilize Seneca Valley Virus (SVV) or SVV derivative in combination with a checkpoint inhibitor, whereby the cancer is refractory to monotherapy with the checkpoint inhibitor.

In one embodiment, the disclosure provides for methods, compositions, kits, and pharmaceutical composition for treating cancer which utilize an SVV derivative encoding a checkpoint inhibitor, whereby the cancer is refractory to monotherapy with the checkpoint inhibitor. In certain embodiments, the SVV derivative encoding a checkpoint inhibitor is used alone or in combination with another checkpoint inhibitor.

In certain embodiments of the disclosure, the cancer may be refractory to both SVV and the checkpoint inhibitor.

The checkpoint inhibitor may be a PD-1 inhibitor, a PD-L1 inhibitor, a CTLA-4 inhibitor, or combinations thereof. In certain embodiments, the checkpoint inhibitor is an anti-PD1-antibody, an anti-PD-L1 antibody, or an anti-CTLA-4 antibody. In certain embodiment, the checkpoint inhibitor blocks one or more of the following checkpoint proteins on cancer cells: PD-1, PD-L1, CTLA-4, B7-1, B7-2 In other embodiments, the checkpoint inhibitor blocks one or more of the following checkpoint proteins: LAG-3; TIM-3; TIGIT; VISTA, B7-H3, BTLA, and Siglec-15. See Qin, S. et al. Mol Cancer 18, 155 (2019); Gaynor et al. Semin Cancer Biol. 2020 Jul. 2; S 1044-579X(20)30152-8. The checkpoint inhibitor may be an antibody such as, a monoclonal antibody.

In certain embodiments, the SVV′ derivative encoding a checkpoint inhibitor encodes more than one checkpoint inhibitor. In certain alternate embodiments, the SVV derivative encoding a checkpoint inhibitor encodes a PD-1 inhibitor, a PD-L1 inhibitor, a CTLA-4 inhibitor, or combinations thereof. In specific embodiments, the SVV derivative encoding a checkpoint inhibitor comprises a nucleic acid encoding an anti-CTLA4 antibody, a nucleic acid encoding an anti-PDL1 antibody, or both. In certain embodiments, the SVV derivative encoding a checkpoint inhibitor comprises a nucleic acid encoding an anti-CTLA4 nanobody, and a nucleic acid encoding an anti-PDL1 nanobody, or both. In certain embodiments, the SVV derivative encoding a checkpoint inhibitor is an SVV derivative disclosed in U.S. Provisional Patent Application No. 63/138,999 filed on Jan. 19, 2021.

Additional exemplary suitable checkpoint inhibitors include but are not limited to ipilimumab (Yervoy®), pembrolizumab (Keytruda®), nivolumab (Opdivo®), and atezolizumab (Tecentriq®). In one embodiment, the checkpoint inhibitor is an anti-PD-1 antibody or nanobody. In another embodiment, the checkpoint inhibitor is an anti-CTLA4 antibody or nanobody.

The cancer treated by the combination of SVV or SVV derivative and checkpoint inhibitor is refractory to monotherapy with the checkpoint inhibitor. Where more than one checkpoint inhibitor is used, the cancer is refractory to monotherapy with at least one of checkpoint inhibitors.

The treatment of cancer provided herein may include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers may include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganglioma, meningioma, adrenocortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Kaposi's sarcoma, moles dysplastic nevi, lipoma, angioma, dermatofibroma, keloids, rhabdomyosarcoma, medulloblastoma, and psoriasis.

In some embodiments, the cancer treated by the presently disclosed methods comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

In other embodiments, the cancer is a neuroblastoma or a melanoma. In yet another embodiment, the cancer is a neuroendocrine cancer or a small cell lung cancer (SCLC) tumor.

In certain embodiments, the combination of the SVV or SVV derivative and the checkpoint inhibitor results in improved cancer treatment when compared to using the SVV alone. In other embodiments, the combination of the SVV or SVV derivative and the checkpoint inhibitor results in improved cancer treatment when compared to using the SVV or SVV derivative or the checkpoint inhibitor alone.

In certain embodiments, the SVV derivative encoding a checkpoint inhibitor results in improved cancer treatment when compared to using the SVV alone. In other embodiments, the SVV derivative encoding a checkpoint inhibitor results in improved cancer treatment when compared to using SVV or the checkpoint inhibitor alone.

### Combination Therapies

The compositions and methods for treating a cancer in a subject using SVV or SVV derivative and a checkpoint inhibitor described herein may be combined with at least one additional compound useful for treating cancer. The compositions and methods for treating a cancer in a subject using a SVV derivative encoding a checkpoint inhibitor described herein may be combined with at least one additional compound useful for treating cancer. The additional compound may comprise a commercially available compound, known to treat, prevent, or reduce the symptoms of cancer and/or metastasis. In certain embodiments, the additional compound may be another checkpoint inhibitor.

In one aspect, the pharmaceutical composition disclosed herein comprises an mTOR inhibitor, an SVV or SVV derivative and a pharmaceutically acceptable carrier. The pharmaceutical composition may be used in combination with a therapeutic agent such as an anti-tumor agent, including, but not limited, to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine.

Other examples of chemotherapeutic agents include, but are not limited to, the following and their pharmaceutically acceptable salts, acids and derivatives: MEK inhibitors, such as but not limited to, refametinib, selumetinib, trametinib or cobimetinib; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK); razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and docetaxel (TAXOTERE, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine: vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, or a combination thereof.

An immunotherapeutic agent may be, but is not limited to, an interleukin-2 or other cytokine, an inhibitor of programmed cell death protein 1 (PD-1) signaling such as a monoclonal antibody that binds to PD-1, Ipilimumab. The immunotherapeutic agent can also block cytotoxic T lymphocytes associated antigen A-4 (CTLA-4) signaling and it can also relate to cancer vaccines and dendritic cell-based therapies.

In one embodiment the subject suffering from cancer is also administered at least one anti-cancer therapeutic agent selected from the group consisting of: a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an engineered anti-cancer virus or virus derivative and a combination of any thereof. In one embodiment, the least one anti-cancer therapeutic agent is administered formerly, simultaneously, or subsequently to the administering of the SVV or SVV derivative.

In one embodiment, the subject is also administered an IFN-I inhibiting agent. The IFN-I inhibiting agent used herein encompasses any agent known in the art for inhibiting, suppressing, or reducing partially or fully and temporarily or permanently IFN type I pathway. In some embodiments, the inhibition effect of the IFN-I inhibiting agent can be reversible. In other embodiments, the inhibition of the IFN-I is reversed.

The inhibiting agent comprises siRNA, ribozyme, an antisense molecule, an aptamer, a peptidomimetic, a small molecule, an mTOR inhibitor, a histone deacetylase (HDAC) inhibitor, a Janus kinase (JAK) inhibitor, an IFN inhibitor, an IFN antibody, an IFN-α Receptor 1 antibody, an IFN-α Receptor 2 antibody and viral peptide and a combination of any thereof. The viral peptide can be, but not limited to, NS1 protein from an Influenza virus or NS2B3 protease complex from dengue virus.

The mTOR pathway and its inhibition are known to be implicated in various diseases such as cancer. Rapamycin is a natural product produced by the bacterium Streptomyces hygroscopicus that can inhibit mTOR through association with its intracellular receptor FK-506 binding protein 12 (FKBP12). The FKBP12-rapamycin complex binds directly to the FKBP12-rapamycin binding domain of mTOR. mTOR functions as a catalytic subunit for two distinct molecular complexes, mTOR complex 1 (mTORC1) and mTOR complex 2 (mTORC2). mTORC1 is composed of regulatory associated protein of mTOR (Raptor) and mammalian LST8/G-protein β-subunit like protein (mLST8/GβL). This complex functions as a nutrient/energy/redox sensor and plays a role in regulating protein synthesis. The activity of mTORC1 is stimulated by insulin, growth factors, serum, phosphatidic acid, amino acids (particularly leucine) and oxidative stress (Hay and Sonenberg, Genes Dev. 18(16):1926-1945, 2004; Wullschleger et al., Cell 124(3):471-484). In contrast, mTORC1 is known to be inhibited by low nutrient levels, growth factor deprivation, reductive stress, caffeine, rapamycin, farnesylthiosalicylic acid and curcumin (Beevers et al., Int. J. Cancer 119(4):757-764, 2006; McMahon et al., Mol. Endocrinol. 19(1):175-183). The components of mTORC2 are rapamycin-insensitive companion of mTOR (Rictor), GβL, mammalian stress-activated protein kinase interacting protein 1 and mTOR. mTORC2 has been shown to function as an important regulator of the cytoskeleton through its stimulation of F-actin stress fibers, paxillin, RhoA, Rac1, Cdc42 and protein kinase C alpha (Sarbassov et al., Curr. Biol. 14(14): 1296-302, 2004; Sarbassov et al., Science 307(5712): 1098-101, 2005). Unlike mTORC1, mTORC2 is not sensitive to rapamycin.

A number of mTOR inhibitors are known in the art and have potent immunosuppressive and anti-tumor activities. Inhibitors of mTOR, such as rapamycin or rapamycin analogs or derivatives, are known to exhibit immunosuppressive and anti-proliferative properties. Other mTOR inhibitors include everolimus, tacrolimus, zotarolimus (ABT-578), pimecrolimus, biolimus, FK-506, PP242 (2-(4-Amino-1-isopropyl-1H-pyrazolo[3,4-d]pyrimidin-3-yl)-1H-indol-5-01), Ku-0063794 (rel-542-[(2R,6S)-2,6-Dimethyl-4-morpholinyl]-4-(4-morpholinyl)pyrido[2,3-d]pyrimidin-7-yl]-2-methoxybenzenemethanol), PI-103 (3-(4-(4-Morpholinyl)pyrido[3′,2′:4,5]furo[3,2-d]pyrimidin-2-yl)phenol), PKI-179 (N-[4-[4-(4-Morpholinyl)-6-(3-oxa-8-azabicyclo[3.2.1]oct-8-yl)-1,3,5-triazin-2-yl]phenyl]-N′-4-pyridinylurea hydrochloride), AZD8055 (542,4-BisR3S)-3-methyl-4-morpholinyl]pyrido[2,3-d]pyrimidin-7-yl]-2-methoxybenzenemethanol), WYE-132/WYE-125132 (1-{4-[1-(1,4-Dioxa-spiro[4.5]dec-8-yl)-4-(8-oxa-3-aza-bicyclo[3.2.1]oct-3-yl)-1H-pyrazolo[3,4-d]pyrimidin-6-yl]-phenyl}-3-methyl-urea), WYE-23 (4-{6-[4-(3-Cyclopropyl-ureido)-phenyl]-4-morpholin-4-yl-pyrazolo[3,4-d]pyrimidin-1-yl}-piperidine-1-carboxylic acid methyl ester), WYE-28 (4-(6-{4-[3-(4-Hydroxymethyl-phenyl)-ureido]-phenyl}-4-morpholin-4-yl-pyrazolo[3,4-d]pyrimidin-1-yl)-piperidine-1-carboxylic acid methyl ester), WYE-354 (4-[6-[4-[(Methoxycarbonyl)amino]phenyl]-4-(4-morpholinyl)-1H-pyrazolo[3,4-d]pyrimidin-1-yl]-1piperidinecarboxylic acid methyl ester), C20-methallylrapamycin and C16-(S)-butylsulfonamidorapamycin, C16-(S)-3-methylindolerapamycin (C16-iRap), C16-(S)-7-methylindolerapamycin (AP21967/C16-AiRap), CCI-779 (temsirolimus), RAD001(40-O-(2-hydroxyethyl)-rapamycin), AP-23575, AP-23675, AP-23573, 20-thiarapamycin, 15-deoxo-19-sulfoxylrapamycin, WYE-592, ILS-920, 7-epi-rapamycin, 7-thiomethyl-rapamycin, (3S,6R,7E,9R,10R,12R,14S,15E,17E,19E,21S,23S,26R,27R,34aS)-9,10,12,13,14,2-1,22,23,24,25,26,27,32,33,34,34a-Hexadecahydro-9,27-dihydroxy-3-[(1R)-2-[(-1S,3R,4R)-3-methoxy-4-tetrazol-1-yl)cyclohexyl]-1-methylethyl]-10,21-dime-t-hoxy-6,8,12,14,20,26-hexamethyl-23,27-epoxy-3H-pyrido[2,1-c][1,4]oxaazac-yc-lohentriacontine-1,5,11,28,29(4H,6H,31H)-pentone) 23,27-Epoxy-3H pyrido[2,1-c] [1,4]oxaazacyclohentriacontine-1,5,11,28,29(4H,6H,31H)-pentone (U.S. Pat. No. 6,015,815), A-94507, Deforolimus, AP-23675, AP-23841, Zotarolimus, CCI779/Temsirolimus, RAD-001/Everolimus, 7-epi-trimethoxy-rapamycin, 2-desmethyl-rapamycin, and 42-O-(2-hydroxy)ethyl-rapamycin, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, 42-O-(2-hydroxy)ethyl rapamycin, ridaforolimus, ABI-009, MK8669, TOP216, TAFA93, TORISEL™ (prodrug), CERTICAN™, Ku-0063794, PP30, Torin1, Torin2, ECO371, AP23102, AP23573, AP23464, AP23841; 40-(2-hydroxyethyl)rapamycin, 40-[3-hydroxy(hydroxymethyl) methylpropanoate]-rapamycin (also called CC1779), 32-deoxorapamycin, and 16-pentynyloxy-32(S)-dihydrorapanycin. Non-rapamycin analog mTOR inhibiting compounds include, but are not limited to, LY294002, wortmannin, quercetin, myricentin, staurosporine, and ATP competitive inhibitors. Other examples of suitable mTOR inhibitors may be found in U.S. Pat. No. 6,329,386, U.S. Publication 2003/129215, and U.S. Publication 2002/123505.

In some embodiments, the disclosed mTOR inhibitor inhibits at least one of mTORC1 and mTORC2. In other embodiments, the disclosed mTOR inhibitor is Torin 1 or Torin 2.

Many HDAC inhibitors are known and used in the art. The most common HDAC inhibitors bind to the zinc-containing catalytic domain of the HDACs. These HDAC inhibitors can be classified into several groupings named according to their chemical structure and the chemical moiety that binds to the zinc ion. Some examples include, but are not limited to, hydroxamic acids or hydroxamates (such as Trichostatin A (TSA) or Vorinostat/SAHA (FDA approved)), aminobenzamides Entinostat (MS-275), Tacedinaline (CI994), and Mocetinostat (MGCD0103), cyclic peptides (Apicidin, Romidepsin (FDA approved)), cyclic tetrapeptides or epoxyketones (such as Trapoxin B), depsipeptides, benzamides, electrophilic ketones, and carboxylic aliphatic acid compounds (such as butyrate, phenylbutyrate, valproate, and valproic acid). Other HDAC inhibitors include, but are not limited to, Belinostat (PXD101), LAQ824, and Panobinostat (LBH589). Examples of HDCA inhibitors in clinical trials include Panobinostat (LBH-589), Belinostat (PXD101), Entinostat (MS275), Mocetinostat (MGCD01030), Givinostat (ITF2357), Practinostat (SB939), Chidamide (CS055/HBI-8000), Quisinostat (JNJ-26481585), Abexinostat (PCI-24781), CHR-3996 and AR-Z2. In one embodiment, the HDAC inhibitor is Trichostatin A.

JAK inhibitors (also referred as JAK/SAT inhibitors) inhibit the activity of one or more of the Janus kinase family of enzymes (e.g. JAK1, JAK2, JAK3, and/or TYK2), thereby interfering with the JAK-STAT signaling pathway. Various JAK inhibitors are known and used in the art for the treatment of inflammatory diseases or cancer. Non-limiting examples of JAK inhibitors are FDA approved compounds including Ruxolitinib (Jakafi/Jakavi), Tofacitinib (Jakvinus, formerly known as tasocitinib and CP-690550), Oclacitinib (Apoquel), Baricitinib (Olumiant, LY3009104), Decernotinib (VX-509). Other JAK inhibitors are under clinical trials and/or used as experimental drugs. These include for instance Filgotinib (G-146034, GLPG-0634), Cerdulatinib (PRT062070), Gandotinib (LY-2784544), Lestaurtinib (CEP-701), Momelotinib (GS-0387, CYT-387), Pacritinib (SB1518), PF-04965842, Upadacitinib (ABT-494), Peficitinib (ASP015K, NJ-54781532), Fedratinib (SAR302503), Cucurbitacin I, CHZ868, ABT-494, dimethyl fumarate (DMF, Tecfidera), GLPG0634, and CEP-33779. In one embodiment, the JAK/STAT inhibitor is staurosporine (STS; antibiotic AM-2282) which is an inhibitor of protein kinase C (PKC).

In one embodiment, the subject is further administered at least one IFN-I inhibiting agent selected from the group consisting of: HDAC inhibitor, JAK/STAT inhibitor, IFN inhibitor, IFN antibody, IFN-α Receptor 1 antibody, IFN-α Receptor 2 antibody and viral peptide and a combination of any thereof. In another embodiment, the at least one IFN-I inhibiting agent is administered formerly, simultaneously, or subsequently to the administering of the SVV or SVV derivative. In some embodiments, the at least one IFN-I inhibiting agent is subsequently removed once the SVV or SVV derivative has replicated in the tumor cells and before the addition of an anti-cancer therapeutic agent (e.g. checkpoint inhibitor).

In one embodiment, the anti-cancer therapeutic agent is administered formerly, simultaneously, or subsequently to the administering of the at least one IFN-I inhibiting agent. In one embodiment, the anti-cancer therapeutic agent is administered subsequently to the administering of the at least one IFN-I inhibiting agent. In another embodiment, the anti-cancer therapeutic agent is administered subsequently to the administering of the at least one IFN-I inhibiting agent and the SVV or SVV derivative.

In one embodiment treatment with the SVV or SVV derivative and the checkpoint inhibitor is preceded by the administration of IFN-I inhibiting agent. In one embodiment, once SVV or SVV derivative replication and cancer cells death are confirmed, the administration of IFN-I inhibiting agent is terminated. For instance, cancer cells can be treated with an IFN-I inhibitor, (e.g. (5-(tetradecyloxy)-2-furoic acid), acetyl-CoA carboxylase inhibitor: TOFA), 24 hours before SVV or SVV derivative treatment and then both treatments can be pursued for several weeks until robust SVV or SVV derivative replication is observed, and markers of cell death are detected. Then the treatment with IFN-I inhibiting agent can be terminated and an anti-cancer therapeutic agent can be initiated. Upon SVV or SVV derivative replication, various nucleic acids and cellular debris are generated which can trigger the activation of an influx of immune cells (e.g. T-cells, NK, cells, APCs, etc.) to proceed in cancer cells' inhibition, reduction and/or elimination/killing. This process of immune response is enhanced further by the termination of IFN-I inhibition.

### Pharmaceutical Compositions

In certain embodiments, the invention is directed to pharmaceutical compositions comprising SVV or SVV derivative and a checkpoint inhibitor. In another embodiment, the invention is directed a pharmaceutical composition comprising SVV or SVV derivative and a separate pharmaceutical composition comprising a checkpoint inhibitor. In yet another embodiment, the invention is directed to a pharmaceutical composition comprising a an SVV derivative encoding a checkpoint inhibitor, which may optionally include a further checkpoint inhibitor.

Provided herein is a pharmaceutical composition for treating a cancer in a subject in need thereof. The pharmaceutical composition comprises a checkpoint inhibitor, an SVV or SVV derivative, and a pharmaceutically acceptable carrier; whereby the cancer is refractory to monotherapy with the checkpoint inhibitor.

Also provided herein is a pharmaceutical composition for treating a cancer in a subject in need thereof. The pharmaceutical composition comprises a checkpoint inhibitor, an SVV or SVV derivative, and a pharmaceutically acceptable carrier; whereby the cancer is refractory to monotherapy with the checkpoint inhibitor.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment provided herein, the pharmaceutical composition useful for practicing the method of the invention may be administered to deliver a dose of between 1 ng/kg/day and 100 mg/kg/day. In another embodiment, the pharmaceutical composition useful for practicing the invention may be administered to deliver a dose of between 1 ng/kg/day and 500 mg/kg/day. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the SVV or SVV derivative can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In one aspect a pharmaceutical composition is disclosed for treating a cancer in a subject. The pharmaceutical composition comprises a checkpoint inhibitor, an SVV or SVV derivative and a pharmaceutically acceptable carrier. In one embodiment, the pharmaceutical composition comprises an SVV derivative encoding a checkpoint inhibitor and a pharmaceutically acceptable carrier. Pharmaceutically acceptable carriers, which are useful, include, but are not limited to, glycerol, water, saline, ethanol, and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers; wetting agents, emulsifiers, salts for influencing osmotic pressure buffers, coloring, flavoring and/or aromatic substances and the like. They may also be combined where desired with other active agents, e.g., other analgesic agents.

The compositions may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof. A particularly preferred preservative is a combination of about 0.5% to 2.0% benzyl alcohol and 0.05% to 0.5% sorbic acid.

The compositions may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition disclosed herein may be used in combination with an additional therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

### Administration/Dosing

In certain embodiments of the invention, the SVV or SVV derivative and the checkpoint inhibitor are administered at the same time. In other embodiments, the checkpoint inhibitor is administered before SVV or SVV derivative is administered. In another embodiment, the checkpoint inhibitor is administered after SVV or SVV derivative administration. In an alternate embodiment, the SVV derivative encoding a checkpoint inhibitor is administered without another checkpoint inhibitor. In other embodiments, the SVV derivative encoding a checkpoint inhibitor is administered with another checkpoint inhibitor, which may be administered, before, after, or concurrently with the SVV derivative encoding a checkpoint inhibitor.

The regimen of administration may affect what constitutes an effective amount. For example, the therapeutic formulations may be administered to the patient subject either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In general, SVV or SVV derivative is administered in an amount of between 10⁷ and 1×10¹¹ vp/kg. The exact dosage to be administered depends on a variety of factors including the age, weight, and sex of the patient, and the size and severity of the tumor being treated.

SVV or SVV derivative is typically administered at a therapeutically effective dose. A therapeutically effective dose refers to that amount of the virus that results in amelioration of symptoms or a prolongation of survival in a patient. Toxicity and therapeutic efficacy of viruses can be determined by standard procedures in cell cultures or experimental animals, e.g., for determining the LD₅₀ (the dose lethal to 50% of the population of animals or cells; for viruses, the dose is in units of vp/kg) and the ED₅₀ (the dose, vp/kg, therapeutically effective in 50% of the population of animals or cells), or the TC₁₀ (the therapeutic concentration or dose allowing inhibition of 50% of tumor cells and can be related to PFU) or the EC₅₀ (the effective concentration, vp/cell, in 50% of the population of animals or cells). The dose ratio between toxic and therapeutic effects is the therapeutic index and it can be expressed as the ratio between LD₅₀ and ED₅₀ or EC₅₀. The dosage of viruses lies preferably within a range of circulating concentrations that include the ED₅₀ or EC₅₀ with little or no toxicity. The dosage may vary within this range depending upon the dosage form employed-and the route of administration utilized.

The SVV or SVV derivative may be present in the composition in multidose and single dosage amounts, including, but not limited to between or between about 1×10⁵ and 1×10¹² pfu, 1λ10⁶ to 1×10¹⁰ pfu, or 1×10⁷ to 1×10¹⁰ pfu, each inclusive, such as at least, or about at least 1×10⁵, 1×10⁶, 1×10⁷, 1×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1×10¹¹, or 1×10¹² pfu.

The volume of the composition can be any volume, and can be for single or multiple dosage administration, including, but not limited to, from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, or 0.5 mL to 5 mL, each inclusive.

The infectivity of the SVV or SVV derivative can be manifested, such as by increased titer or half-life of the oncolytic virus when exposed to a bodily fluid, such as blood or serum. Infectivity can be increased by any amount, including, but not limited to, at least 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold. 6-fold, 7-fold, 8-fold, 9-fold, or 10-fold.

Administration of the compositions of the present invention to a patient subject, preferably a mammal, more preferably a human, may be carried out using known procedures, at dosages and for periods of time effective to treat cancer in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound is from about 0.01 to about 50 mg/kg of body weight/per day.

The SVV or SVV derivative and the checkpoint inhibitor can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for treating cancer in a patient.

### Routes of Administration

One skilled in the art will recognize that although more than one route can be used for administration, a particular route can provide a more immediate and more effective reaction than another route. In certain embodiments of the invention, the SVV or SVV derivative and the checkpoint inhibitor are administered via the same route of administration. In other embodiments, the SVV or SVV derivative and checkpoint inhibitor are administered via different routes of administration.

In one embodiment, the SVV or SVV derivative is administered intratumorally. In another embodiment, the checkpoint inhibitor is administered systemically. In an alternate embodiment, the SVV or SVV derivative is administered intratumorally and the checkpoint inhibitor is administered systemically.

Routes of administration of the disclosed compositions (containing SVV or SVV derivative and/or the checkpoint inhibitor) include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder, or aerosolized formulations for inhalation, compositions, and formulations for intravesical administration and the like. It should be understood that the formulations and compositions that would be useful in the present invention are not limited to the particular formulations and compositions that are described herein. In one embodiment, the SVV or SVV derivative treatment and/or treatment with the checkpoint inhibitor comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal, and any combination thereof.

### Kits

The invention also includes kits containing the SVV or SVV derivative and a checkpoint inhibitor, whereby the kits are used to treat a cancer that is refractory to monotherapy with the checkpoint inhibitor.

In further embodiments a kit is provided for treating or ameliorating a cancer, as described elsewhere herein wherein the kit comprises: a) SVV or SVV derivative or composition comprising SVV or SVV derivative; b) a checkpoint inhibitor; and optionally c) an additional agent or therapy as described herein. The kit can further include instructions or a label for using the kit to treat or ameliorate the cancer. The kit can also include an assay to confirm that the cancer is indeed refractory to the checkpoint inhibitor. In yet other embodiments, the invention extends to kits assays for a given cancer (such as, but not limited to, small-cell Lung cancer or triple negative breast cancer), as described herein. Such kits may, for example, contain the reagents from PCR or other nucleic acid hybridization technology (microarrays) or reagents for immunologically based detection techniques (e.g., ELISpot, ELISA.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### EXAMPLE 1: Combination Therapy Using SVV and Checkpoint Inhibitor in Cancers Refractory to Checkpoint Inhibitors

Seneca Valley Virus (SVV-001) is an oncolytic virus in the picornaviridae family that to date has only been tested as a single dose intravenous monotherapy in early clinical trials of patients having cancers with neuroendocrine properties (e.g., NET, NEC). Recent data in the oncolytic and immunotherapy fields have demonstrated that oncolytic viruses can enhance efficacy in preclinical models and in clinical trials when the oncolytic virus agent was injected intratumorally in combination with a systemic administration of a checkpoint inhibitor (CPI). Two murine syngeneic tumor models of neuroblastoma and melanoma origin that are resistant to CPI therapy were tested. When injected intra-tumorally in these immune competent animal models, SVV-001 reversed resistance to CPI and enhanced efficacy of checkpoint inhibitors in both tumor models.

SVV-001 has been identified as an extremely tumor selective and potent oncolytic virus against human tumors. It has recently been discovered that the receptor for SVV is TEM8, a protein that is highly and specifically expressed in multiple cell types within solid tumors. Interestingly, TEM8 is expressed in malignant cancer cells and cancer stem cells and also in “normal” cells in the tumor microenvironment, including, angiogenic endothelial cells, cancer associated fibroblasts, and pericytes. SVV infects cancer cells and cancer stem cells while it is unknown if SVV infects or otherwise inhibits growth of these additional critical cell types of the tumor microenvironment. SVV-001 possesses a number of features that make it an exemplary oncolytic virus, namely, its ability to target and penetrate solid tumors due to its extremely small (27 nM) size, specificity toward tumor cells mediated by TEM8 expression, the ability to easily manufacture, and arm SVV-001 with anti-tumor transgenes and the inability of this virus for insertional mutagenesis. SVV-001 has been studied in both pediatric and adult early phase studies reporting safety (one DLT reported in 76 patients due to tumor pain) and shows promising signs of efficacy in patients.

The addition of immune checkpoint inhibitor (CPI) therapies can substantially augment anti-tumor effects in various cancers; however, neuroendocrine cancers and small cell lung cancer (SCLC) tumors have proven refractory to CPI monotherapy. Using the murine N1E-115 neuroblastoma and the B16F10 metastatic melanoma model engineered to express the TEM8 receptor, the effect of combining SVV-001 and CPI therapy using a checkpoint inhibitor (anti-PD-1 antibodies) was evaluated. Both murine lines are resistant to anti-PD-1 treatment. N1E-115 cells endogenously express high levels of TEM8 whereas B16F10 does not and is thus resistant to SVV. Transfecting TEM8 into B16F10 causes the cells to now become susceptible to SVV infection.

SVV-001 was injected twice weekly as monotherapy or in combination with murine anti-PD1 administered twice weekly. The types of cells infected, immune infiltrate, SVV replication, and immune responses were examined along with efficacy. SVV increased the response rate 3-6-fold over the CPI alone (p<0.01) or 6-fold over SVV-001 monotherapy (p<0.01) and improved survival in animals treated with the combination therapy. Thus, two syngeneic murine models for SVV-001 immunotherapy were developed.

This Example also shows that the combination of SVV-001 and anti-PD-1 provides a significant improvement in anti-tumor response. The studies in this Example may serve as a foundation for translating SVV-001 oncolytic virotherapy combined with anti-PD-1 antibodies in patients with neuroendocrine tumors. Thus, this Example establishes that SVV overcomes resistance to checkpoint inhibitor therapies in neuroendocrine and melanoma murine models.

### EXAMPLE 2: Combination Therapy Using SVV and Checkpoint Inhibitor

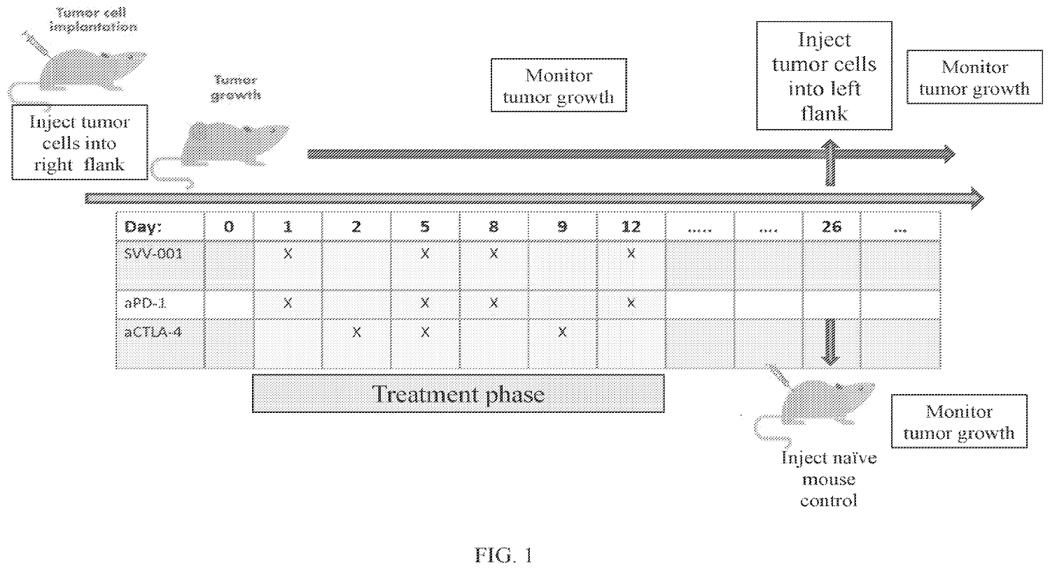

A further study investigating combination therapy of SVV and various checkpoint inhibitors was carried out. The Pan02 Tumor Model Treatment schedule for this study is shown in FIG. 1.

Study Outline:

Pan02 pancreatic syngeneic tumor cells were implanted into right flank of syngeneic C57BL/6 mice. When tumors were 100 mm³, treatment was initiated with SVV +/−anti-PD-1; anti-CTLA4 for 4 injections (days 1-12). On day 26, Pan02 tumor cells were injected into the left flank and also into naïve control mice. Tumor growth and survival were monitored. The study used murine antibodies anti-PD-1 (RMP1-14) and anti-CTLA-4 (9D9)-analogs of Nivolumab and Ipilimumab (BMS).

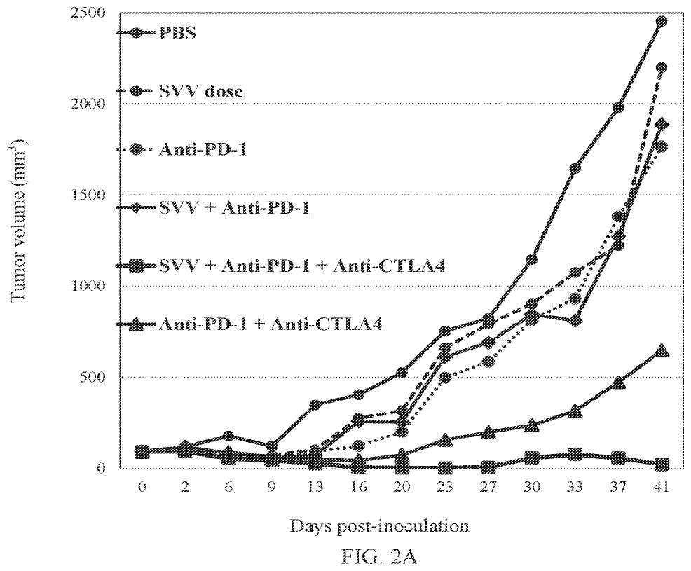

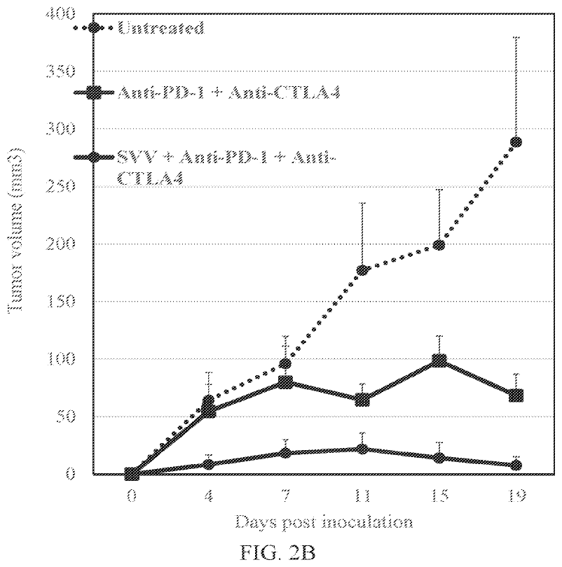

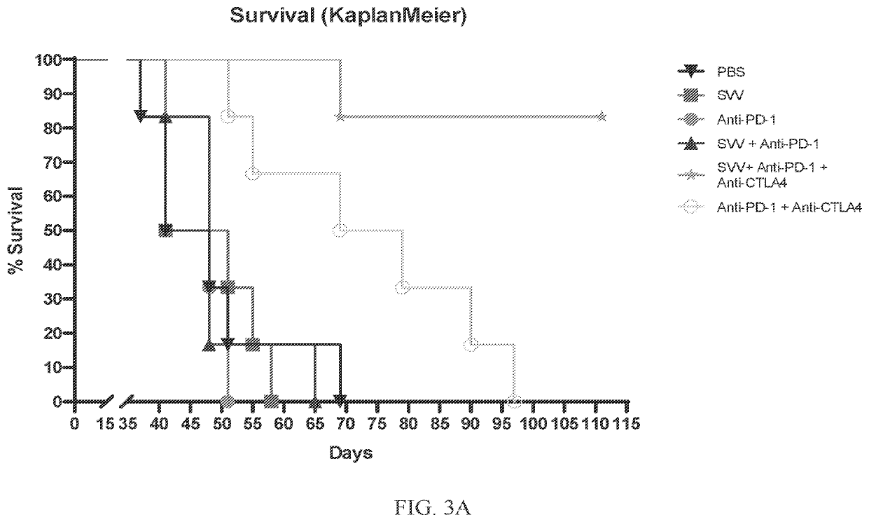

The results of this study are shown in FIGS. 2A, 2B, 3A, 3B, 3C and 4. The effects of administering SVV, checkpoint inhibitors and SVV and checkpoint inhibits in primary tumors are shown in FIG. 2. 5/6 cures were observed in the SVV+aPD1+aCTLA4 group.

The effects of administering SVV, checkpoint inhibitors and SVV and checkpoint inhibits in contralateral tumors tumor are shown in FIG. 2B. Again, 5/6 cures were observed in the SVV+aPD1+aCTLA4 group. As shown in FIG. 2B, in contralateral tumors the combination of SVV, anti-PD-1 and anti-CTLA4 result in 83% cures. There were no cures for the control or for the checkpoint inhibitors administered alone.

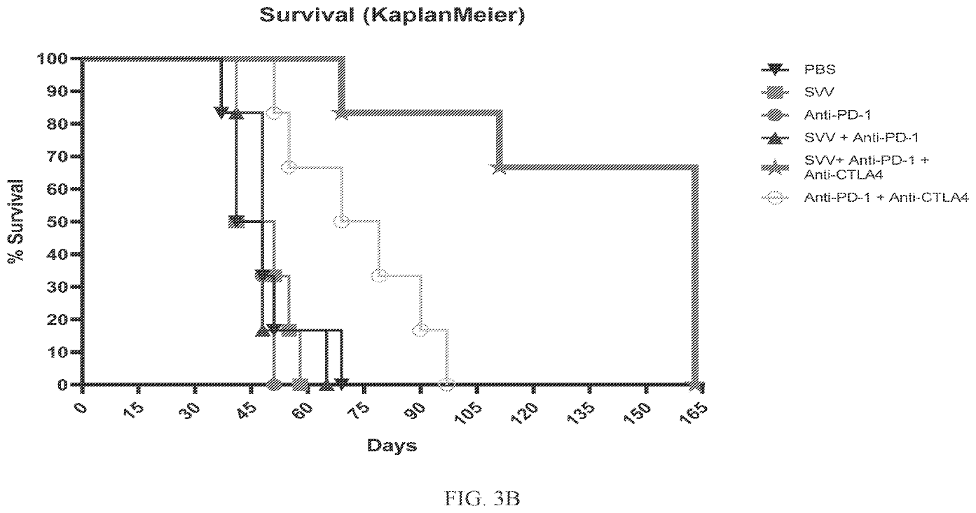

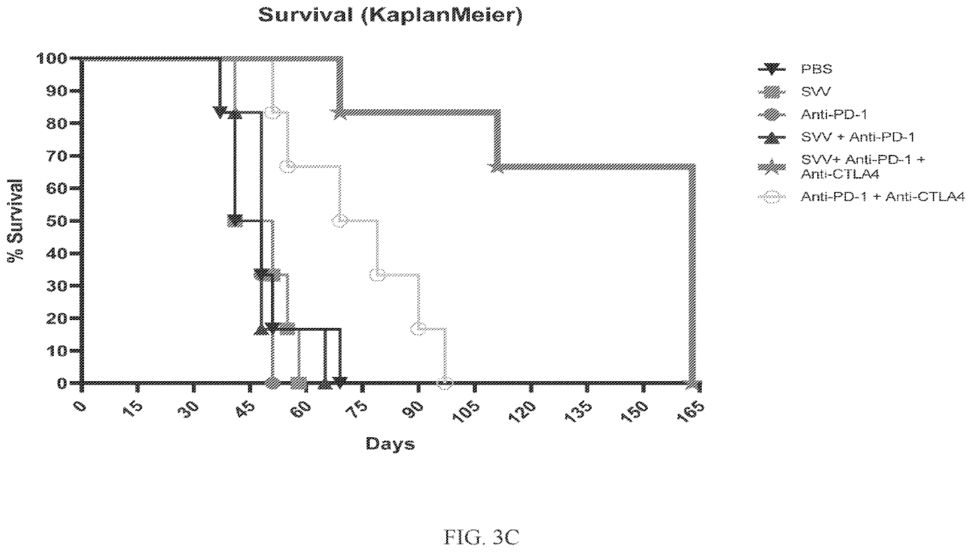

A Kaplan Meier plot depicting the percentage survival of cells as a function of time after treatment with SVV, checkpoint inhibitors or combinations thereof is shown in FIG. 3A and FIG. 3B. FIG. 3A shows the results up to 115 day. FIG. 3B and FIG. 3C show the results up to 165 days. All of the remaining animals were sacrificed on day 165. As shown in FIGS. 3A-3C, SVV+aPD1+aCTLA4 produce long term cures.

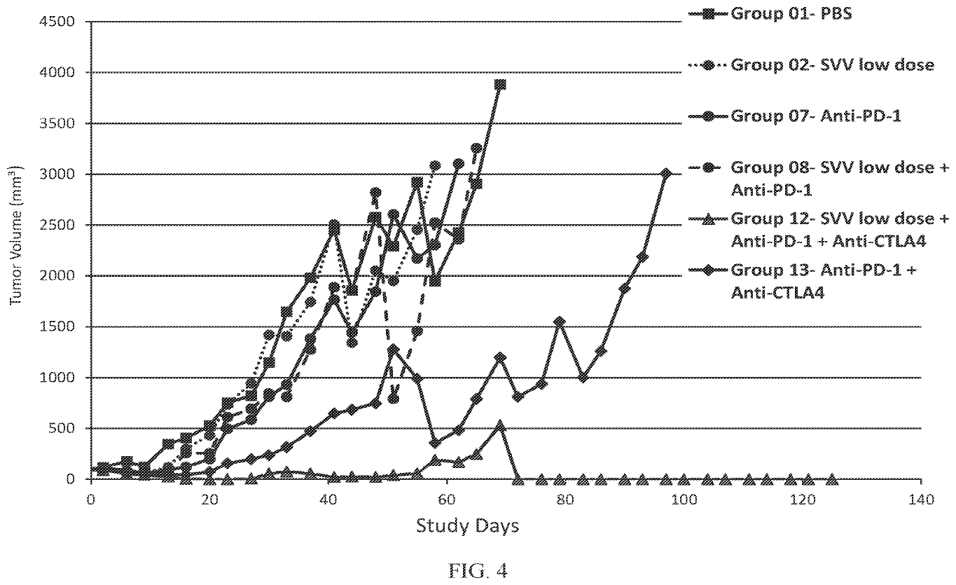

As is evident from the plot, the combination of SVV+anti-PD-1 and anti-CTL4 resulted in the longest survival. The tumor volume after treatment with SVV, checkpoint inhibitors or combinations thereof as a function of time is shown in FIG. 4. SVV-001 treatment stopped at day 12. Anti-PD1/anti-CTLA4 treatment stopped at day 23.

Observations:

SVV monotherapy and SVV+anti-PD1 show an effect during the treatment period (to day 13); after this, these tumors grow similarly to PBS-treated mice (i.e. the tumors have become refractive to treatment with the virus).

Anti-PD1+anti-CTLA4 combinations shows tumor growth control for about 16-20 days and then tumors grow, but more slowly than PBS control. SVV+anti-PD1+anti-CTLA4 (S+P+C) combination shows tumor regressions and cures by day 20. Four mice showed tumor growth by day 33 and then 3 regressed to cures. Overall, 5 or 6 mice showed durable cures to day 120. The anti-PD1+anti-CTLA4 (P+C) group showed delayed tumor growth but no long-term cures. In contralateral tumors, 5 or 6 mice showed complete regression in S+P+C group whereas only 1 of 6 showed regression in P+C group. The S+P+C group mice are still alive and tumor-free at day 145+.

### EXAMPLE 3: Combination Therapy Using SVV and Checkpoint Inhibitor

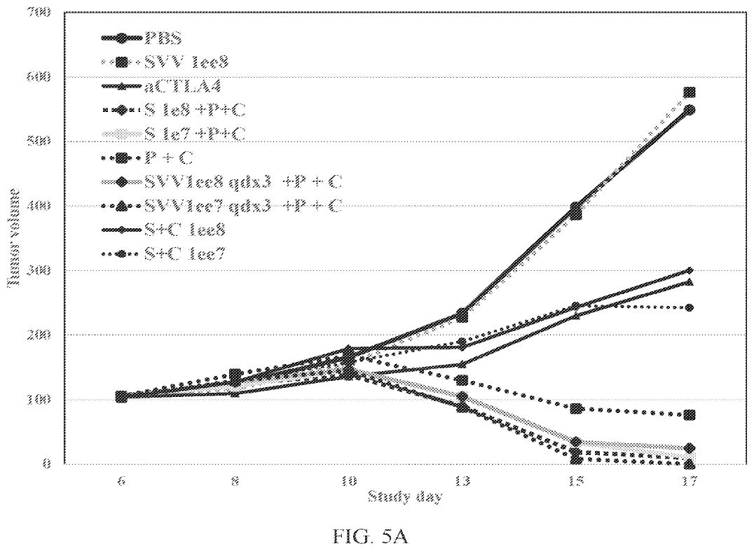

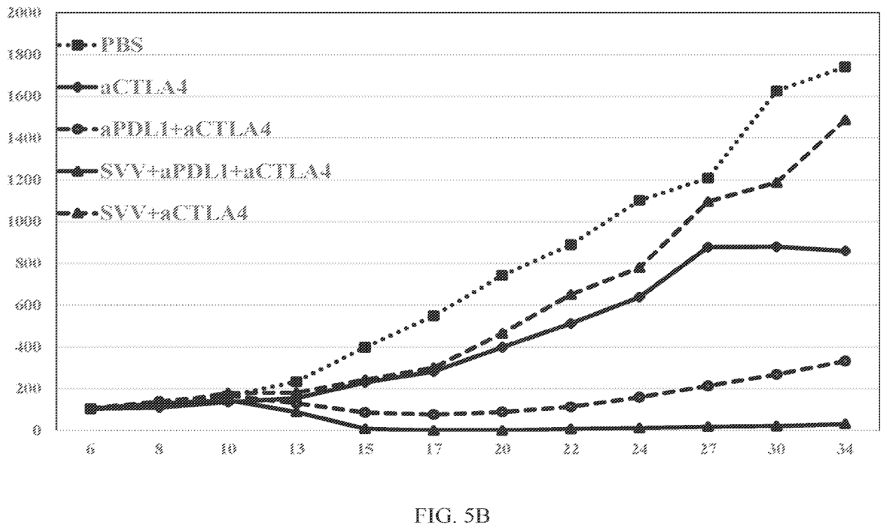

A further studying using the protocol of Example 2 was carried out. FIG. 5A and FIG. 5B show the tumor volume after treatment SVV, checkpoint inhibitors or combinations thereof. FIG. 5A shows the data until day 17. FIG. 5B shows the data until day 34.

The efficacy for this study was similar to the study shown in Example 2. S+P+C causes tumor regression/cures but P+C only shows tumor reductions. SVV+anti-CTLA4 did not produce any cures and has similar effect to anti-CTLA4 alone. Anti-PD1+anti-CTLA4 produced 2 of 8 cures. S+P+C produces 5-7 of 8 cures (depending on treatment group).

### ILLUSTRATIVE EMBODIMENTS

Provided here are illustrative embodiments of the disclosed technology. These embodiments are illustrative only and do not limit the scope of the present disclosure or of the claims attached.

Embodiment 1. A method of treating a cancer in a subject in need thereof comprising administering to the subject an effective amount of a Seneca Valley Virus (SVV) or SVV derivative and an effective amount of a checkpoint inhibitor, wherein the cancer is refractory to monotherapy with the checkpoint inhibitor.

Embodiment 2. The method of embodiment 1, wherein the cancer is also refractory to monotherapy with SVV.

Embodiment 3. The method of embodiment 1, wherein the checkpoint inhibitor is a PD-1 inhibitor, a PD-L1 inhibitor, a CTLA-4 inhibitor, or a combination thereof.

Embodiment 4. The method of embodiment 2, wherein the method comprises administering a PDL-1 inhibitor and a CTLA-4 inhibitor.

Embodiment 5. The method of embodiment 1, wherein the method comprises administering an SVV derivative encoding the checkpoint inhibitor.

Embodiment 6. The method of embodiment 4, wherein the SVV derivative encodes a PD-1 inhibitor, a CTLA-4 inhibitor, or both.

Embodiment 7. The method of embodiment 4, wherein the method comprises administering an additional checkpoint inhibitor.

Embodiment 8. The method of embodiment 3, wherein the checkpoint inhibitor is an antibody or nanobody.

Embodiment 9. The method of embodiment 1, wherein the checkpoint inhibitor is an anti-PD-1 antibody.

Embodiment 10. The method of embodiment 1, wherein the method comprises administering an anti-PD-1 antibody and an anti-CTLA-4 antibody.

Embodiment 11. The method of any one of embodiments 1-10, wherein the checkpoint inhibitor is administered before, concurrent or after administration of the Seneca Valley Virus (SVV) or SVV derivative.

Embodiment 12. The method of any one of embodiments 1-11, wherein the Seneca Valley Virus (SVV) or SVV derivative is administered intratumorally.

Embodiment 13. The method of any one of embodiments 1-12, wherein the checkpoint inhibitor is administered systemically.

Embodiment 14. The method of any one of embodiments 1-12, wherein the Seneca Valley Virus (SVV) or SVV derivative is administered intratumorally and wherein the checkpoint inhibitor is administered systemically.

Embodiment 15. The method of any one of embodiments 1-14, wherein the treatment is improved compared to monotherapy with Seneca Valley Virus (SVV) or SVV derivative or the checkpoint inhibitor.

Embodiment 16. The method of any one of embodiments 1-15, wherein the Seneca Valley Virus (SVV) or SVV derivative and checkpoint inhibitor are administered at the same administration interval.

Embodiment 17. The method of embodiment 11, wherein the Seneca Valley Virus (SVV) or SVV derivative and checkpoint inhibitor are administered weekly.

Embodiment 18. The method of any one of embodiments 1 to 18, wherein the cancer is a neuroblastoma or a melanoma.

Embodiment 19. The method of any one of embodiments 1 to 18, wherein the cancer is a neuroendocrine cancer or a small cell lung cancer (SCLC) tumor.

Embodiment 20. The method of any one of embodiments 1 to 18 wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Embodiment 21. A pharmaceutical composition for treating a cancer in a subject, the pharmaceutical composition comprising a checkpoint inhibitor, an SVV or SVV derivative, and a pharmaceutically acceptable carrier, wherein the cancer is refractory to monotherapy with the checkpoint inhibitor.

Embodiment 22. The pharmaceutical composition of embodiment 21, wherein the cancer is also refractory to monotherapy with SVV.

Embodiment 23. The pharmaceutical composition of embodiments 21 or 22, wherein the checkpoint inhibitor is a PD-1 inhibitor, a PD-L1 inhibitor, or a CTLA-4 inhibitor.

Embodiment 24. The pharmaceutical composition of embodiment 23, wherein the checkpoint inhibitor is an antibody or nanobody.

Embodiment 25. The pharmaceutical composition of embodiment 21, wherein the checkpoint inhibitor is an anti-PD-1 antibody.

Embodiment 26. The pharmaceutical composition of any one of embodiments 21-23, wherein the composition comprises an SVV derivative encoding the checkpoint inhibitor.

Embodiment 27. The pharmaceutical composition of embodiment 26, wherein the SVV derivative encodes a PD-1 inhibitor, a CTLA-4 inhibitor, or both.

Embodiment 28. The pharmaceutical composition of any one of embodiments 21-26, wherein the composition comprises an anti-PD-1 antibody and an anti-CTLA-4.

Embodiment 29. The pharmaceutical composition of any one of embodiments 21 to 28, wherein the cancer is a neuroblastoma or a melanoma.

Embodiment 30. The pharmaceutical composition of any one of embodiments 21 to 21, wherein the cancer is a neuroendocrine cancer or a small cell lung cancer (SCLC) tumor.

Embodiment 31. The pharmaceutical composition of one of any one of embodiments 21 to 28, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Embodiment 32. A kit for treating cancer in a subject comprising a Seneca Valley Virus (SVV) or SVV derivative combined with a checkpoint inhibitor, wherein the cancer is refractory to monotherapy with the checkpoint inhibitor.

Embodiment 33. The kit of embodiment 32, wherein the checkpoint inhibitor is a PD-1 inhibitor, a PD-L1 inhibitor, or a CTLA-4 inhibitor.

Embodiment 34. The kit of embodiment 32, wherein the checkpoint inhibitor is an antibody or nanobody.

Embodiment 35. The kit according to embodiment 32, wherein the kit comprises an SVV derivative encoding the checkpoint inhibitor.

Embodiment 36. The kit according to embodiment 32, wherein the SVV derivative encodes a PD-1 inhibitor, a CTLA-4 inhibitor, or both.

Embodiment 37. The kit of any one of embodiments 32-36, wherein the cancer is a neuroblastoma, a melanoma, a neuroendocrine cancer, or a small cell lung cancer (SCLC) tumor.

Embodiment 38. The kit of any one of embodiments 32-36, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Embodiment 39. A Seneca Valley Virus (SVV) or SVV derivative (SVV) in combination with a checkpoint inhibitor for use in the manufacture of a medicament for treatment of a cancer, wherein the cancer is refractory to monotherapy with the checkpoint inhibitor.

Embodiment 40. The combination of embodiment 39, wherein the checkpoint inhibitor is a PD-1 inhibitor, a PD-L1 inhibitor, or a CTLA-4 inhibitor.

Embodiment 41. The combination of embodiment 39, wherein the checkpoint inhibitor is an antibody or nanobody.

Embodiment 42. The combination of embodiment 39, wherein the SVV derivative encodes the checkpoint inhibitor.

Embodiment 43. The combination of embodiment 42, wherein the SVV derivative encodes a PD-1 inhibitor, a CTLA-4 inhibitor, or both.

Embodiment 44. The combination of any one of embodiments 39-43, wherein the cancer is a neuroblastoma, a melanoma, a neuroendocrine cancer, or a small cell lung cancer (SCLC) tumor.

Embodiment 45. The combination of any one of embodiments 39-43, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

It is to be understood that while the disclosure has been described in conjunction with the preferred specific embodiments thereof, that the foregoing description and the examples that follow are intended to illustrate and not limit the scope of the disclosure. It will be understood by those skilled in the art that various changes may be made, and equivalents may be substituted without departing from the scope of the disclosure, and further that other aspects, advantages and modifications will be apparent to those skilled in the art to which the disclosure pertains. In addition to the embodiments described herein, the present disclosure contemplates and claims those inventions resulting from the combination of features of the disclosure cited herein and those of the cited prior art references which complement the features of the present disclosure. Similarly, it will be appreciated that any described material, feature, or article may be used in combination with any other material, feature, or article, and such combinations are considered within the scope of this disclosure.

The disclosures of each patent, patent application, and publication cited or described herein are hereby incorporated herein by reference, each in its entirety, for all purposes.

1. A method of treating a cancer in a subject in need thereof comprising administering to the subject an effective amount of a Seneca Valley Virus (SVV) or SVV derivative and an effective amount of a checkpoint inhibitor, wherein the cancer is refractory to monotherapy with the checkpoint inhibitor.

2. The method of claim 1, wherein the cancer is also refractory to monotherapy with SVV.

3. The method of claim 1, wherein the checkpoint inhibitor is a PD-1 inhibitor, a PD-L1 inhibitor, a CTLA-4 inhibitor, or a combination thereof.

4. The method of claim 2, wherein the method comprises administering a PDL-1 inhibitor and a CTLA-4 inhibitor.

5. The method of claim 1, wherein the method comprises administering an SVV derivative encoding the checkpoint inhibitor.

6. The method of claim 4, wherein the SVV derivative encodes a PD-1 inhibitor, a CTLA-4 inhibitor, or both.

7. The method of claim 4, wherein the method comprises administering an additional checkpoint inhibitor.

8. The method of claim 3, wherein the checkpoint inhibitor is an antibody or nanobody.

9. The method of claim 1, wherein the checkpoint inhibitor is an anti-PD-1 antibody.

10. The method of claim 1, wherein the method comprises administering an anti-PD-1 antibody and an anti-CTLA-4 antibody.

11. The method of claim 1, wherein the checkpoint inhibitor is administered before, concurrent or after administration of the Seneca Valley Virus (SVV) or SVV derivative.

12. The method of claim 1, wherein the Seneca Valley Virus (SVV) or SVV derivative is administered intratumorally.

13. The method of claim 1, wherein the checkpoint inhibitor is administered systemically.

14.-15. (canceled)

16. The method of claim 1, wherein the Seneca Valley Virus (SVV) or SVV derivative and checkpoint inhibitor are administered at the same administration interval.

17. The method of claim 11, wherein the Seneca Valley Virus (SVV) or SVV derivative and checkpoint inhibitor are administered weekly.

18.-19. (canceled)

20. The method of claim 1, wherein the cancer comprises a triple negative breast cancer, a small cell lung cancer, a non-small cell lung cancer, a non-small cell squamous carcinoma, an adenocarcinoma, a glioblastoma, a neuroblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

21. A pharmaceutical composition for treating a cancer in a subject, the pharmaceutical composition comprising a checkpoint inhibitor, an SVV or SVV derivative, and a pharmaceutically acceptable carrier, wherein the cancer is refractory to monotherapy with the checkpoint inhibitor.

22. The pharmaceutical composition of claim 21, wherein the cancer is also refractory to monotherapy with SVV.

23. The pharmaceutical composition of claim 21, wherein the checkpoint inhibitor is a PD-1 inhibitor, a PD-L1 inhibitor, or a CTLA-4 inhibitor.

24. The pharmaceutical composition of claim 23, wherein the checkpoint inhibitor is an antibody or nanobody.

25. The pharmaceutical composition of claim 21, wherein the checkpoint inhibitor is an anti-PD-1 antibody.

26. The pharmaceutical composition of claim 21, wherein the composition comprises an SVV derivative encoding the checkpoint inhibitor.

27. The pharmaceutical composition of claim 26, wherein the SVV derivative encodes a PD-1 inhibitor, a CTLA-4 inhibitor, or both.

28. The pharmaceutical composition of claim 21, wherein the composition comprises an anti-PD-1 antibody and an anti-CTLA-4 antibody.

29.-30. (canceled)

31. The pharmaceutical composition of one of any one of claims 21 to 28, wherein the cancer comprises a triple negative breast cancer, a small cell lung cancer, a non-small cell lung cancer, a non-small cell squamous carcinoma, an adenocarcinoma, a glioblastoma, a neuroblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

32. A kit for treating cancer in a subject comprising a Seneca Valley Virus (SVV) or SVV derivative combined with a checkpoint inhibitor, wherein the cancer is refractory to monotherapy with the checkpoint inhibitor.

33. The kit of claim 32, wherein the checkpoint inhibitor is a PD-1 inhibitor, a PD-L1 inhibitor, or a CTLA-4 inhibitor.

34. The kit of claim 32, wherein the checkpoint inhibitor is an antibody or nanobody.

35. The kit according to claim 32, wherein the kit comprises an SVV derivative encoding the checkpoint inhibitor.

36. The kit according to claim 32, wherein the SVV derivative encodes a PD-1 inhibitor, a CTLA-4 inhibitor, or both.

37. (canceled)

38. The kit of claim 32, wherein the cancer comprises a triple negative breast cancer, a small cell lung cancer, a non-small cell lung cancer, a non-small cell squamous carcinoma, an adenocarcinoma, a glioblastoma, a neuroblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

39.-45. (canceled)
